(19) United States
(12) Patent Application Publication
(10) Pub. No.: US 2006/0264921 A1
(43) Pub. Date: Nov. 23, 2006

(54) Retractable Surgical Instruments

(75) Inventors: Harvey Deutsch, Los Angeles, CA (US); Anthony Stephen Kewitsch, Santa Monica, CA (US)

Correspondence Address: ANTHONY KEWITSCH, 515 OCEAN AVE., UNIT 505-S, SANTA MONICA, CA, 90402, US

(73) Assignee: IMFLUX LLC, Santa Monica (US)

(21) Appl. No.: 11/306,208

(22) Filed: Dec. 20, 2005

Related U.S. Application Data
(60) Provisional application No. 60/640,698, filed on Dec. 29, 2004.

(51) Int. Cl. A61B 18/18

(52) U.S. Cl. 606/32

(57) ABSTRACT

An electrosurgical instrument system for ready deployment of the instrument at the end of a line within a sterile surgical field employs a disposable reel detachably mounted on a base unit which allows smooth manual withdrawal and provides controlled motor-driven retraction of the line and instrument. The line includes power, control and ground circuits and the circuits are completed through a reel in a detachable cassette mounted on a base unit, which includes a tension-controlled belt permitting manual payout and controlled drive return of the line and instrument.

### REFERENCE TO RELATED APPLICATIONS

This application relies for priority on previously filed provisional application entitled “Retractable Surgical Probe”, Ser. No. 60/640,698, filed on Dec. 29, 2004.

### FIELD OF THE INVENTION

This invention relates to electrosurgical systems using electrosurgical tools to cut and coagulate tissue, and more particularly to devices and methods to manipulate electrosurgical cable within a sterile surgical field easily and in a manner to minimize the risk of patient burns.

### BACKGROUND OF THE INVENTION

The technique of electrosurgery involves the application of a relatively high voltage, radio frequency (RF) electrical signal to a localized region of the patient during surgery, for the purposes of cutting tissue, coagulating blood flow from tissue, or simultaneously cutting and coagulating tissue. Electrosurgical instruments come in various forms, such as pencils, laproscopic probes, electrosurgical scissors, electrosurgical forceps, etc. Electrosurgical devices generally fall into two categories, monopolar and bipolar, depending on the electrical circuit configuration. In a monopolar device a radio frequency signal is supplied to an active electrode, which is used to affect tissue at the target site. The contact area of the return electrode is sufficiently large that no burning occurs at this location. This return electrode completes the electrical circuit to the patient at a location far from the target site. In contrast, in a bipolar arrangement both the active and return electrodes are present on the handpiece, and current flows from the active electrode to the return electrode, by way of an arc formed there between. In both approaches, the sinusoidal RF electrical signal is created by an electrosurgical generator, and the relatively small area of tissue in the vicinity or contact with the electrode provides an intense localized electrical current density which produces the cutting or coagulation action. Monopolar and bipolar electrosurgery both introduce unique requirements related to delivering high voltage RF energy to the electrosurgical instrument in a safe manner.

Electrosurgical instruments are commonly used in “keyhole” or minimally invasive surgery and also in “open” surgery. A surgeon controls the delivery of power to the handpiece by depressing a finger switch on the handpiece, or by stepping on a footswitch which activates the handpiece, which delivers an activation signal to the electrosurgical generator. The electrosurgical generator responds to the activation signal by delivering the high voltage RF power to the active electrode of the handpiece. Typically there are two settings for the electrosurgical handpiece, for cutting or coagulating or both. There may be two finger switches on the handpiece to select the mode of operation. As a result, the cable attached to the electrosurgical generator and a monopolar electrosurgical handpiece consists of three parallel conductors, one providing RF power and the other two providing the cutting and coagulation activation signals back to the generator. Bipolar cables have in addition a return conductor. Conventional electrosurgical cables must provide conductors having sufficient diameters to safely conduct the electrosurgical power produced by the generator without dissipating excessive power. The cables must be flexible and compact and be sufficiently well insulated to prevent shorting of the high voltage RF signal.

Various safety, ease-of-use and cost improvements relating to electrosurgical instruments have been disclosed in the prior art. Three enhancements which have significantly improved the safety of electrosurgical procedures include the replacement of the ground referenced return electrode with a “floating” return electrode, active electrode monitoring and return electrode monitoring. For example, EP 1374788A1 by Fleming et al. describes an electrosurgical generator which analyzes the impedance of the electrode circuit, and interrupts the radio frequency signal when the rate of change of impedance increases, indicating the onset of a “flare-out”. In this way, the power is reduced before flare-out occurs, preventing injury to the patient or failure of the instrument. U.S. Pat. No. 5,376,089 by Smith describes an electrosurgical handpiece with a cable comprised of a main contact wire attached to the electrosurgical probe and two switch wires, which conduct when the corresponding button is pushed. When a user pushes one of two buttons, the instrument provides a cutting or coagulating current. U.S. Pat. No. 6,190,385 describes an electrical cable and connectors optimized for bipolar electrosurgery. U.S. Patent Application 2004/0097117 by Gonnering describes a monopolar electrosurgical multi-plug connector device providing greater convenience.

Despite the effort to enhance the inherent safety during electrosurgery, a significant number of electrosurgical related accidents, such as patient and/or doctor burns or even the outbreak of fire within the surgery room, still occur. Two recommended techniques to prevent these accidents include the removal of electrosurgical pencils or electrodes from the center of the surgical field between uses and the storage of the electrosurgical pencil in a holster between uses. The present design of electrosurgical pencil systems do not adequately provide this functionality.

Spring powered cable retraction units are available in the electronics field to take up excess lengths of cable; however, such units must be attached to a fixed point such that the retraction unit does not simply drag when the cable is extended in opposition to the power spring. To prevent this, present approaches of clipping the cable to the curtain are inadequate. A rigid “fixed point” at a central location within the surgical field is not available. In addition, spring retraction mechanisms exhibit poor control of the withdrawal speed during retraction. This may cause the pencil to exhibit significant backlash upon retraction. It is also difficult to initiate retraction of the pencil into its holster from a remote location outside of the surgical field. Therefore, an inexpensive and convenient approach to electrosurgical cable management has not yet been developed.

### SUMMARY OF THE INVENTION

An electrosurgical system for use under sterile conditions includes a base unit mountable within the surgical field near an operating table, and supporting a detachable cassette including an electrosurgical instrument and attached feed cable that can be manipulated within the sterile field. The multi-line cable is wound about a disposable reel and has a free end coupled to the electrosurgical instrument and a fixed central end lending to plug-in connectors along the rotating axis of the reel. The operative components are sterile as they enter the surgical field and the base may be additionally protected from contamination by a disposable cover and an interface element insertable between the base unit and the cassette. The system allows the instrument and cable to be smoothly withdrawn manually to any desired length from the reel during surgery, after which the instrument and cable can be withdrawn by motor drive under surgeon control onto the reel. Then the reel, cable, and electrosurgical instrument can be detached as desired for replacement by a new sterile unit.

In accordance with the invention, the base unit is an enclosed structure with an plug-in top mount for the reel on a drive hub rotatable about a vertical axis. The plug-in mount receives electrical bayonet connectors extending from the bottom of the reel and attached to the fixed end of the multi-line cable wrapped about the reel hub. The free end of the cable couples to the electrosurgical instrument, such as a Bovie knife, which may for convenience be kept in an accessible holder. The electrical circuits from the cable extend centrally within the drive hub, and include rotary electrical couplings in the form of a number of conductive rings at different levels for each of the lines of the cable. External contacts electronically connect each of the conductive rings to different circuits, providing control signals for instrument energization, motor power drive, and ground connections. Within the housing, a drive motor responsive to control signals turns a drive capstan which is coupled by a drive belt to the drive hub for the reel. The drive belt normally is under sufficiently low tension to allow the surgeon to pay out the multi-cable line manually in smooth and easy fashion. In order to retract the line and the instrument, a control on the instrument (or a foot pedal) is engaged, and the motor is actuated while a solenoid shifts a moveable roller so that the tension on the drive belt increases and the drive hub is rotated to retract the cable at a controlled rate.

This arrangement also can include other attractive features from the standpoint of sterile surgical use, because the replaceable cassette can be isolated from the base unit by an adapter plate inserted between the plug-in connectors and the sterile shroud covering the base unit. In addition, the entire housing and support structure can conveniently be mounted for spatial flexibility on a gooseneck support engaged to the surgical table.

A feature of the invention resides in the detachable rotary coupling for multi-line cable and reel, with the lines of the cable coupling via bayonet style prongs into the center core of the reel hub to couple individually to different slip ring contacts. The entire module, including the electrosurgical instrument, is of sufficiently low cost to allow disposal after a single use.

In this invention, therefore we disclose an electrosurgical retraction unit which withdraws the electrosurgical cable and pencil between use in a simple and cost-effective manner. Typically, the electrosurgical cable and the electrosurgical pencil holster are clipped to the sterile drape covering the patient and the top of the operating table. The length of cable needed to extend the pencil from the holster to the surgical site is placed on top of the drape. It is advantageous to develop a device which retracts this length of cable into an enclosure between each use. This reduces the risk of capacitive coupling between excess cable lengths and the patient. In addition, it is further advantageous that a nurse outside of the surgical field can remotely activate the retraction between each use, to assist the surgeon in maintaining an orderly work area.

### BRIEF DESCRIPTION OF THE DRAWINGS

A better understanding of the invention may be had by reference to the following description, taken in conjunction with the accompanying drawings in which:

FIG. 1 is a perspective view, partially broken away and exploded, of an assembly in accordance with the invention utilizing both a disposable reel and electrosurgical instrument, and a support and drive housing mounted on a support attachable to a surgical table as used in relation to a sterile surgical field;

FIG. 2 is a cross sectional view in exploded form of a disposable reel and the electrical coupling between the cable on the reeel and the rotating shaft and electrical slip rings;

FIG. 3 is top cutaway perspective view of the system detailing the drive coupling between the rotating shaft and drive motor;

FIG. 4 is another cutway view of the interior of the base unit and detachable reel, showing further details thereof, and

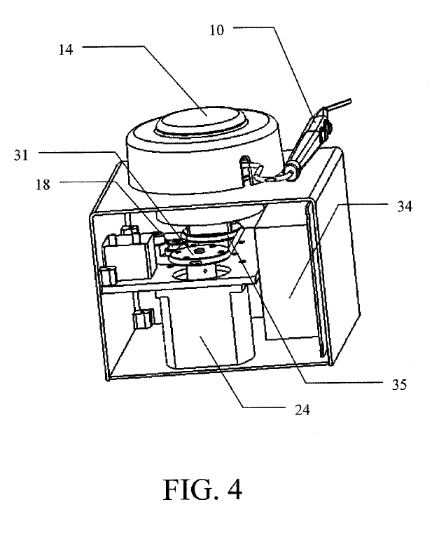

FIG. 5 is a perspective view of the electrosurgical instrument held within a mating interface on cassette housing.

### DETAILED DESCRIPTION OF THE INVENTION

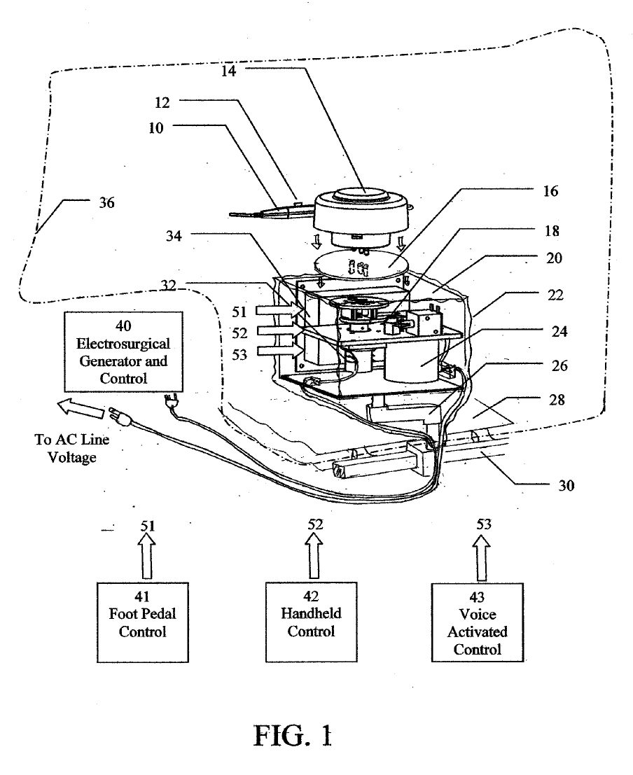

As seen in FIG. 1 a sterile surgical field 36 (outlined by dotted lines) above an operating table 28 defines a volume which must be protected against contamination before and during the conduct of surgical procedures. The surgical table 28 is covered with sterile sheeting to which elements and lines (not shown) may be clipped into place as needed in conventional fashion which need not be further described in detail. In accordance with the invention, an electrosurgical retraction base unit 20 is mounted in proximity to the surgical field, here supported on a gooseneck mount 26 which adds a further position adjustment capability. The base unit 20 is covered by a sterile shroud 22 which allows access, at a top side plug in receptacle 25, for a plug-in cassette 14 including a reel 15 on which is wound a multi-line cable 11. The cable 11 couples at its free end to an electrosurgical instrument 10, such as a Bovie knife in this instance. The fixed end of the cable 11 is diverted to the central hollow core of the reel 15 and is terminated in connector body 13.

In the preferred embodiment, a “Bovie” type electrosurgical instrument, consisting of a pencil-like probe 10 with cutting and coagulation activation buttons 12 (only one of which is seen) and removable active electrode, is attached to the power supply 40 through a three wire, parallel conductor cable 11. The Bovie device includes buttons to energize the active electrode tip for cutting and/or coagulating. An electrosurgical system incorporating such a pencil is illustrated in FIG. 1. The cable 11 to the electrosurgical pencil 10 is retracted into the cable cassette 14 by winding about a rotating reel 15. One end of the electrosurgical cable 11 is terminated to connector 13 contacts integrated into the rotating spool 15. The base unit 20, whose position is adjustable and lockable by use of a semi-rigid gooseneck attachment 26, attaches the entire base unit 20 to a support bar 30 attached to surgical table 28. The unit 20 is held fixed in place within the surgical field 36 and in the vicinity of the patient, but which can be manually positioned by applying a force which causes inelastic deformation. During normal use, the gooseneck experiences forces which are not sufficient to cause inelastic deformation, so that its shape is preserved. For instance, the gooseneck 26 is designed to have sufficient rigidity to maintain the position of the pod even as the cable is retracted or extended. The base unit 20 is also interfaced to a source of ac line voltage, to an electrosurgical generator 40, and potentially to a remote foot switch 41, hand-held remote control 42, or a voice-activated control 43. The remote controls 41, 42, 43 may be achieved by a wired or wireless electronic interfaces (not shown in detail) 51, 52, 53, respectively. Since the gooseneck and base unit extend into the surgical field, sterility must be maintained. Therefore, the sterile, disposable flexible plastic sleeve 22 slides over the base unit 20 and gooseneck 26. Furthermore, a disposable sterile interface disk 16 maintains sterility of the rotating plug-in receptacle 25 of the drive unit even when the cassette 14 is interchanged during a surgical procedure. The cassette and Bovie pencils themselves are sterile and disposable.

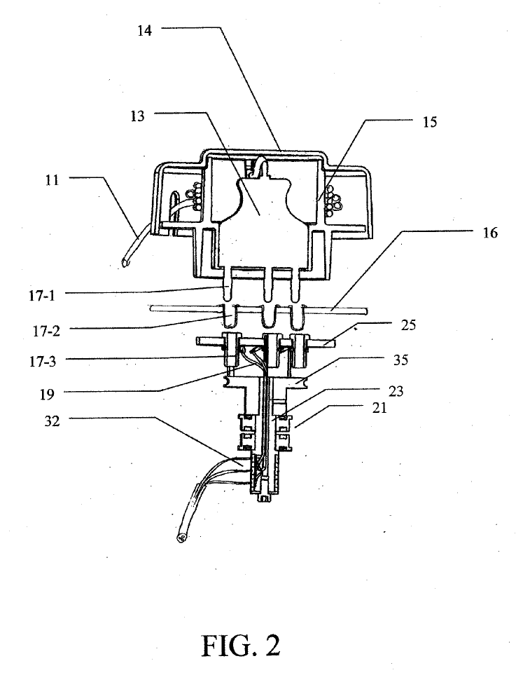

FIG. 2 illustrates a cross sectional view of the cable cassette 14 in detail, consisting of a rotating spool 15 for winding of cable 11. When the motor unit 24 powers the rotary shaft 23, the spool 15 rewinds the cable 11. FIG. 2 shows further details in cross section of the rotating shaft 23 including a plug-in receptacle 25 including electrical contacts 17-3 which mate with the contacts of reel 17-1 through the contacts 17-2 of a sterile isolation plate 16. Contacts 17-3 are connected to wires 19 which attach to rotary shaft coupling 32. Bearings 21 maintain receptacle 25 in fixed alignment with the central axis during rotation.

This invention addresses the unique constraints imposed by the sterility and safety requirements demanded in surgical procedures. In particular, during a surgical procedure, the work area surrounding the patient is clearly demarcated by a “surgical field,” (36 in FIG. 1) in which strict sterilization procedures are enforced. Surgeons follow scrubbing procedures to minimize the risk of infection before entering the surgical field and must repeat the scrubbing procedure should they exit and re-enter the surgical field. In addition, all instruments used within the surgical field are to be sterilized. Due to the complex nature of surgical procedures, the field 36 can become cluttered with a wide variety of instruments and extended cables (e.g., 11) which the surgeon may only use intermittently.

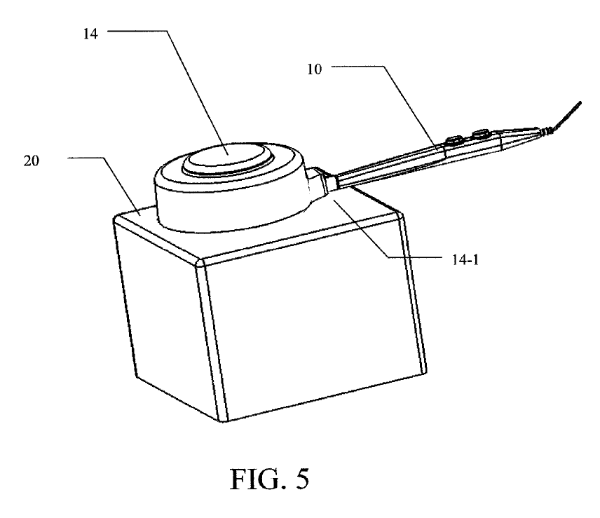

To provide an organized and safe operating environment, it is advantageous to provide electrosurgical instruments 10 whose cable can be retracted into a cassette 14. In addition, the instrument 10 can be retracted so that it returns to a cassette holder section 14-1 formed in cassette body (FIG. 5). The cable and cassette must be sterile, as they extend into the surgical field 36. Typically, sterility is achieved by the design of low cost devices which can be made disposable. The electrosurgical system described herein achieves the added functionality disclosed herein by using a combination of disposable, sterile and non-disposable, non-sterile elements. In particular, a low cost disposable cassette is used with a non-disposable drive and control unit. The drive unit, namely the base unit 20 and gooseneck 26, are reusable. The disposable cassette 14 includes a plastic sleeve (not shown) which covers the reel 15 and gooseneck to isolate them from the sterile surroundings. The electrosurgical generator 40 and foot switch 41 are maintained outside the surgical field and do not have sterility requirements. Handheld control 42 may or may not be maintained within the surgical field.

The cassette further includes a guide and holder 14-1 so that the pencil docks with the cassette when it is fully retracted (FIG. 5). This is accomplished by including a cavity which is sized such that the base of the electrosurgical hand-piece mates with the cassette. In the preferred embodiment the cable cassette is fabricated of injection molded plastic, wherein the housing consists of upper and lower shells which are chemically or ultrasonically bonded, joined with screws or with a friction lock mechanism. The cable 11 preferentially consists of three individually insulated, parallel copper conductors within an outer insulation tube with outer diameter of approximately 3 mm. The gauges of the wires are sufficiently high such that the cable maintains adequate flexibility while still providing low resistance.

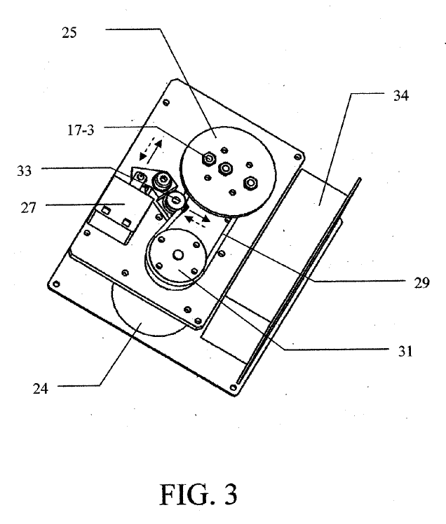

The cable 11 can be pulled from the cassette 14 by disengaging the spool 15 and mandrel shaft 23 from the drive hub 31 so it can freely rotate. This is achieved by way of a clutch mechanism within the base unit 20. The clutch is engaged or disengaged based on the controller state. For example, in FIG. 3, a solenoid 27 engages idler pulley 33 with drive belt 29 to tension drive belt 29 so that it engages motor drive hub 31 with drive capstan 35 located beneath the plug-in receptacle 25 at the upper surface. This action is represented by the solid arrows in FIG. 3. The motor 24 is disengaged from the drive capstan 35 by allowing the solenoid 27 to disengage idler pulley 33 from belt 29. This action is represented by the dashed arrows in FIG. 3. When the cable 11 is not being retracted the drive belt 29 is allowed to slip. To initiate retraction of the cable, the user activates one of the controllers 41, 42, and or 43 to tension the drive belt 29 and activate the motor 24.

The required length of cable to be retracted determines the outer diameter of the spiraled cable through straightforward geometrical considerations. This length can be calculated for a spiral of maximum diameter D with a core of diameter d and wire of thickness t. For typical applications, the total length of cable to be retracted is 2 meters. The typical outer diameter of electrosurgical cable is 3 mm (so t=6 mm) and core diameter d is 25 mm. For these parameters and a double layer spool, the diameter D of the spiral is is 68 mm. This diameter can be reduced by incorporating a spool of added height to make room for multiple layers of windings. FIG. 2, for example, illustrates a spool with four layers of winding.

In other examples, this approach to introduce a sterile operative element into a surgical field, whether or not using a flexible gooseneck, can be employed with a broad range of surgical instruments. For example, the base uint may be used to distribute illumination by way of light guides, suction/vacuum/gas/respirator lines by way of flexible tubing, and EKG or other sensor connections by way of wires. In addition, the use of disposable cassettes to distribute varying lengths of wire, tubing, etc. into the surgical field reduces clutter and the potential for accidents. A gooseneck or other mount may be set in place and fixed in position, or it may be remotely actuated like a robotic arm such that the position of the base unit in its total of six positional and angular degrees of freedom can be set.

Those skilled in the art will readily observe that numerous modifications and alterations of the device may be made while retaining the teachings of the invention. Accordingly, the above disclosure should be construed as limited only by the metes and bounds of the appended claims.

What is claimed is:

1. An assembly for enabling convenient surgical use of a sterile electrosurgical instrument within a sterile surgical field, comprising:
a drive assembly including a housing to be mounted within the surgical field, the assembly including a drive hub in the housing, the drive hub being rotatable about a vertical axis and including engageable rotary electrical connectors therein;
a supply reel including multi-line cable windable thereon, and having an end in fixed relation to a central region of the reel, and an electrosurgical instrument coupled to a free end of the cable;
an electrical engagement mechanism in the drive hub for detachably coupling the multi-line cable to the rotary electrical connectors;
the drive assembly further including a drive capstan in the housing and rotatable about an axis parallel to and spaced apart from the drive hub axis, a drive motor responsive to control signals coupled to the drive capstan, a drive belt engaging about the peripheries of the drive capstan and the drive hub in nominally low friction engagement;
a solenoid actuated idler mechanism responsive to control signals and positioned within the housing against the drive belt to tension the drive belt against the drive capstan and drive hub when in an engaged mode, and to allow manual rotation of the drive hub and supply reel when in the non-engaged mode; and
a control system providing control signals to the drive motor, the solenoid mechanism, and the medical instrument.

2. An instrument as set forth in claim 1 above, wherein the supply reel has an interior cavity for receiving a fixed end of the multi-line cable, and includes multiple turns of cable adapted to be wrapped around the reel to the instrument at the free end thereof, the multiple lines including at least one control line and at least one power line for electrical power to the instrument, and a source of electrical energy in the housing responsive to control signals and coupled to the power lines.

3. An instrument as set forth in claim 2 above wherein the control system includes an operator controllable element on the instrument and an operator controllable foot pedal, both coupled to the circuits in the drive assembly; and the control system responds to the foot pedal and the instrument control circuit for providing control signals for the instrument, the drive motor, and the solenoid actuated idler.

4. An instrument as set forth in claim 3 above, wherein the supply reel includes a plug-in connector extending from the bottom thereof and connecting to the multi-line cable, and wherein the drive hub includes a connector matingly receiving the plug-in connector from the supply reel and a multi-line section extending therefrom into the drive hub at a rotary contact region, and wherein the rotary electrical connectors include rotary contacts at different levels relative to the drive hub axis, and peripheral contact elements in electrical circuit at each of the different levels.

5. An instrument as set forth in claim 4 above, wherein the rotary electrical connectors comprise a group of conductive contact discs alternating with insulator discs, and wherein the peripheral contact elements comprise elongated spring elements disposed to engage individual different conductive discs, and wherein the instrument further includes a detachable isolation plate disposed between the mating plug-in connectors.

6. An instrument as set forth in claim 5 above, wherein the drive hub includes a hollow interior extending from the connector to the rotary electrical connectors, and wherein the multi-line cable joins the connector to the different conductive disks, and wherein the drive belt is disposed in a plane transverse to the longitudinal axes of the drive motor and the drive hub.

7. An instrument as set forth in claim 6 above, wherein the solenoid actuated idler mechanism comprises an arm coupled to the solenoid, and wherein the solenoid when engaged pivots the arm to engage the roller against the belt.

8. An attachment for use with an electrosurgical instrument comprising the combination of a reel with a center hollow hub and including an electro-mechanical coupling extending parallel to the central axis of the hub for engaging a drive system, a multi-line cable wrapped about the hub and including an electrosurgical instrument and a fixed end disposed through the hollow hub and engaged to the coupling, whereby when the reel is engaged it provides both mechanical engagement for the reel and electrical engagement for the lines.

9. A device as set forth in claim 8 above, wherein the reel, cable and instrument are sterile and disposable.

10. A device as set forth in claim 9 above, wherein the electro-mechanical coupling includes multiple male connectors depending from the hub, and wherein the cable fixed end on the hub extends from the exterior of the reel to the hollow interior thereof to join the electro-mechanical coupling.

11. A device as set forth in claim 10 above, further including a drive unit having a drive hub with an upper surface configured to receive the electro-mechanical coupling from the reel, and having a rotary electrical coupling disposed below the reel area, the drive hub having an open hollow interior and including electrical connections from the inserted electro-mechanical coupling to the rotary electrical coupling, and wherein the device further includes a rotary drive coupling engaged to the drive hub.

12. A device as set forth in claim 11 above, including a rotary drive device spaced apart from the drive hub and disposed about an axis parallel thereto, and an engagement mechanism for coupling the drive device to the drive hub.

13. A device as set forth in claim 12 above, wherein the drive unit includes a sterile cover open at the upper end of the rotary drive unit, and an interface sterile cover disposed over the opening in the base unit cover and rotatable with the drive hub.

14. An electrosurgical instrument system for supplying and retrieving an electrosurgical instrument and attached cable within a surgical field comprising:
a reel and a cable wound thereon for providing energy for an instrument attached thereto, the reel including central conductors for the cable in a central region thereof; and
a base unit including a drive hub with an upper surface including mating conductors accessible to the conductors of the reel, the base unit including a rotary drive and a drive coupling engaging the rotary drive selectively to the drive hub for allowing (1) manual rotation of the drive hub and cassette for supplying cable and (2) power driven retraction of the cable.

15. An electrosurgical system in accordance with claim 14 wherein the reel and cable wound thereon are housed within a detachable cassette.

16. An electrosurgical system in accordance with claim 15 wherein the cassette has connector means to provide temporary electrical and mechanical coupling with base unit.

17. An electrosurgical system in accordance with claim 16 wherein the cassette is sterile, disposable and attached to a non-sterile base unit partially covered by a sterile shroud through a sterile interface plate including connector means providing temporary electrical and mechanical coupling of cassette to base unit without compromising sterility of the cassette.
